(19) United States
(12) Patent Application Publication
(10) Pub. No.: US 2025/0352730 A1
(43) Pub. Date: Nov. 20, 2025

(54) VACUUM PRESSURE SYRINGE ASSEMBLY WITH LOCKING FEATURE

(71) Applicant: Becton, Dickinson and Company, Franklin Lakes, NJ (US)

(72) Inventors: Manish Kumar, Bengaluru (IN); Praveen Nalawade, Belgaum (IN); Karthik MR, Bengaluru (IN); Shishir Prasad, Ramsey, NJ (US); Ajay Sakpal, Boonton Township, NJ (US)

(73) Assignee: Becton, Dickinson and Company, Franklin Lakes, NJ (US)

(21) Appl. No.: 18/663,394

(22) Filed: May 14, 2024

(51) Int. Cl. A61M 5/20; A61M 5/31; A61M 5/315

(52) U.S. Cl. CPC A61M 5/2053; A61M 5/3135; A61M 5/3137; A61M 5/31515

(57) ABSTRACT

A syringe assembly comprises a syringe barrel having an open proximal end having a pair of gripping flanges and a distal end including a distal wall having a passageway therethrough in fluid communication with the chamber, the open proximal end including a peripheral flange. An elongate plunger rod is disposed within the syringe barrel, the elongate plunger rod comprising a distal end and a proximal end, the distal end including a stopper, the elongate plunger rod including a plurality of slots located between the distal end and the proximal end, and the elongate plunger rod extendible to a plurality of different locked lengths using a locking element mounted to the proximal end of the syringe barrel to engage the plurality of plunger rod locking slots to permit varying amounts of fluid to be aspirated into the syringe barrel without rotating the elongate plunger rod.

### TECHNICAL FIELD

The present disclosure generally relates to syringe assemblies with a locking feature. More particularly, embodiments of the disclosure provide syringe assemblies with a locking feature that are useful in removing obstructions from blood vessels, for example, removal of plaque and clots from coronary arteries.

### BACKGROUND

Various procedures use vacuum pressure syringes to suction blockages from blood vessels. These procedures include thrombectomies, which is a procedure to remove blood clots from arteries and veins using a syringe and a catheter. Angioplasty is a procedure to open blocked arteries, also using a syringe and a catheter to remove plaque blocking an artery. Existing syringes used for these procedures include a plunger rod having locking fins on the plunger rod to lock the syringe after retracting the syringe and creating a sustained vacuum in an attached catheter or tubing set that has been inserted into a patients' blood vessel to aspirate fluid from the blood vessel. Locking of the syringe requires rotation of the plunger rod.

FIG. 1 shows another type of existing vacuum pressure syringe 10 comprising a syringe barrel 12, a plunger rod 14, a stopper 16 that attaches to the plunger rod. A locking ring 18 engages an inner surface 20 of the syringe barrel and an inner surface 22 of the locking ring engages the plunger rod. One or more features 21 on the inner surface 20 of the syringe barrel engage the locking ring 18, and the plunger rod is rotated to lock the plunger rod 14 in variety of positions to lock the syringe after retracting the syringe and creating a sustained vacuum in an attached catheter or tubing set that has been inserted into a patients' blood vessel. A disadvantage of existing vacuum pressure syringes is that there is a need to rotate the plunger before locking the device, which can be cumbersome and difficult to accomplish. There is a need to provide improved vacuum pressure syringes used in suction and aspiration procedures that are less cumbersome and easier to lock and unlock.

### SUMMARY

Embodiments of the disclosure pertain to a syringe assembly comprising a syringe barrel having a side wall with an inside surface defining a chamber configured to retain and expel a fluid, an open proximal end having a pair of gripping flanges and pair of connecting flanges extending radially from the open proximal end and a distal end including a distal wall having a passageway therethrough in fluid communication with the chamber, the open proximal end including a peripheral flange. The syringe assembly further comprises an elongate plunger rod disposed within the syringe barrel, the elongate plunger rod comprising a distal portion and a proximal portion, the distal end including a stopper and a proximal end, the plunger rod including a plurality of plunger rod locking slots located between the distal end and the proximal end, and the elongate plunger rod extendible to a plurality of different locked lengths using a locking element mounted to the proximal end of the syringe barrel to engage the plurality of plunger rod locking slots to permit varying amounts of fluid to be aspirated into the syringe barrel without rotating the elongate plunger rod.

In a second embodiment, the locking element comprising a clip assembly slidably mounted to the connecting flanges, the clip assembly comprising a main body having locking slot that slidably engages the plurality of plunger rod locking slots by a pushing force.

In a third embodiment, the locking element comprises an elongate bracket including a distal end and a proximal end slidably mounted to the distal end of the syringe barrel.

In additional embodiments, a tamper-evident or tamper proof feature is included with the syringe assembly.

### BRIEF DESCRIPTION OF THE DRAWINGS

Exemplary embodiments of the disclosure are further described in the following detailed description in conjunction with the accompanying drawings, in which:

FIG. 1 is an exploded isometric view of a prior art locking syringe;

FIG. 2 is an isometric view of a first embodiment of syringe assembly;

FIG. 3A is an isometric partial view of the syringe assembly shown in FIG. 2 in an unlocked position;

FIG. 3B is an isometric partial view of the syringe assembly shown in FIG. 2 in locked position;

FIG. 4 is an exploded view of the syringe assembly shown in FIG. 2;

FIG. 5A is a side elevation view of the syringe assembly shown in FIG. 2;

FIG. 5B is a distal end view of the syringe assembly shown in FIG. 2 in an unlocked position;

FIG. 5C is a side elevation of the syringe assembly shown in FIG. 2 with the elongate plunger rod partially retracted from the syringe barrel with fluid aspirated in the syringe barrel;

FIG. 5D is a side elevation of the syringe assembly shown in FIG. 50 after the fluid has been expelled from the syringe assembly;

FIG. 5E is an isometric view of the syringe assembly shown in FIG. 5C in a locked position;

FIG. 5F is a distal end view of the syringe assembly shown in FIG. 50 after the fluid has been expelled from the syringe assembly in a locked position;

FIG. 6 is an isometric view of a second embodiment of syringe assembly;

FIG. 7 is a rear isometric partial view of the syringe assembly shown in FIG. 6 in a locked position;

FIG. 8A is an isometric view of an elongate plunger rod of the syringe assembly shown in FIG. 6;

FIG. 8B is an isometric view of a locking pin of the syringe assembly shown in FIG. 6;

FIG. 8C shows a coil spring of the syringe assembly shown in FIG. 6.

FIG. 8D shows a locator pin of the syringe assembly shown in FIG. 6;

FIG. 8E shows a locking element comprising a clip assembly of the syringe assembly shown in FIG. 6;

FIG. 9 is a side elevation view showing operation of the locking element of the syringe assembly shown in FIG. 6 in an unlocked position;

FIG. 9B is a side elevation view showing operation of the locking element of the syringe assembly shown in FIG. 6 in a locked position;

FIG. 10 shows the locking element of the syringe assembly shown in FIG. 6 prior to assembly to the syringe barrel;

FIG. 11 shows the locking element of the syringe assembly shown in FIG. 6 assembled to the syringe barrel;

FIG. 12 is an isometric view of a third embodiment of syringe assembly;

FIG. 13A is an isometric view of the syringe assembly shown in FIG. 12 with the elongate plunger rod retracted from the syringe barrel;

FIG. 13A is an isometric view of the syringe assembly shown in FIG. 12 the elongate plunger rod inserted in the syringe barrel;

FIG. 14A shows a side elevation view of the syringe assembly shown in FIG. 12 in a locked position;

FIG. 14B is an enlarged view showing the locking portion of the syringe assembly shown in FIG. 12;

FIGS. 15A-B show a tamper-evident feature configured for use with the syringe assembly shown in FIG. 2;

FIG. 15C shows the tamper evident feature of FIGS. 15A-B in the unused position;

FIG. 15D shows the tamper evident feature of FIGS. 15A-B in the used position;

FIGS. 15E-F show the tamper evident feature of FIGS. 15A-B indicating that the syringe assembly has been used;

FIGS. 16A-B show side elevation views of a tamper-evident feature of a syringe assembly configured for use with the syringe assembly shown in FIG. 6;

FIG. 16C shows an end view of the tamper evident feature shown in FIGS. 16A-B;

FIG. 16D shows a top plan view of the tamper evident feature shown in FIGS. 16A-B;

FIG. 16E shows a side elevation view of the syringe assembly shown after the syringe assembly has been used;

FIG. 16F shows the clip re-applied to the syringe assembly shown in FIGS. 16A-B;

FIGS. 17A-B show a side elevation view of a tamper evident label for a syringe assembly according to one or more embodiments;

FIGS. 17C-D show the syringe assembly shown in FIGS. 17A-B indicating that the syringe assembly has been used; and

FIG. 17E shows the tamper evident label re-applied to the syringe assembly after use.

To facilitate understanding, identical reference numerals have been used, where possible, to designate identical elements that are common to the figures. The figures are not drawn to scale.

### DETAILED DESCRIPTION

In one embodiment, slots in plunger ribs in a linear pattern along the syringe axis to interlock with a bracket which locks the syringe and movement of plunger. Vacuum is maintained with locking of the plunger. There is also a locking bracket to engage with slots in the plunger to lock the syringe. The locking bracket is pivoted on the non-gripping flange area of barrel. The locking bracket has a finger support to swing the bracket to lock with the plunger and swing back to unlock the syringe. Since the locking bracket is attached to the non-gripping flange area of the syringe barrel, it will not impact the user experience and the syringe can be handled in the same way as currently. In the locked position, the plunger can't move in and out of the syringe barrel, and in the unlocked position, the plunger is permitted to move in and out of the syringe barrel.

Before describing several exemplary embodiments of the disclosure, it is to be understood that the disclosure is not limited to the details of construction or process steps set forth in the following description. The disclosure is capable of other embodiments and of being practiced or being conducted in many ways.

In this disclosure, a convention is followed wherein the distal end of the device is the end closest to a patient and the proximal end of the device is the end away from the patient and closest to a practitioner.

With respect to terms used in this disclosure, the following definitions are provided. As used herein, the use of “a,” “an,” and “the” includes the singular and plural.

As used herein, the term “Luer connector” refers to a connection collar that is the standard way of attaching syringes, catheters, hubbed needles, IV tubes, etc. to each other. In some embodiments, the Luer connector comprises a luer slip connector including male and female interlocking tubes (a male luer slip connector and a female luer slip connector), slightly tapered to hold together better with even just a simple pressure/twist fit. Luer connectors can optionally include an additional outer rim of threading, allowing them to be more secure. The Luer connector male end is generally associated with a flush syringe and can interlock and connect to the female end located on the vascular access device (VAD) Luer connector also has a distal end channel that releasably attaches the Luer connector to the hub of a VAD, and a proximal end channel that releasably attaches the Luer connector to the syringe barrel of a syringe.

Referring now to FIG. 2, an isometric view of a first embodiment of syringe assembly 100 is shown. The syringe assembly 100 includes a syringe barrel 102 having a side wall 104 with an inside surface defining a chamber 106 configured to retain and expel a fluid, an open proximal end 109 having a pair of gripping flanges 110, 112 and a pair of connecting flanges 111, 113 extending radially from the open proximal end 109 and a distal end 115 including a distal wall 116 having a passageway 118 therethrough in fluid communication with the chamber 106. In FIG. 2, the distal end 115 of the syringe barrel includes a collar 117, which may include a Luer connector or other suitable connection configured to make a connection to a suitable device such as tubing, a catheter or a system for removing fluid from a blood vessel.

The syringe assembly 100 further includes an elongate plunger rod 120 disposed within the syringe barrel 102, the elongate plunger rod 120 comprising a distal end 121 and a proximal end 122, the distal end 121 including a stopper 124, the elongate plunger rod including a plurality of plunger rod locking slots 126 located between the distal end 121 and the proximal end 122, and the elongate plunger rod 120 extendible to a plurality of different locked lengths using a locking element 130 mounted to the proximal end 109 of the syringe barrel 102 to engage the plurality of plunger rod locking slots 126 to permit varying amounts of fluid 140 to be aspirated into the syringe barrel 102 without rotating the elongate plunger rod 120. The proximal end 122 of the elongate plunger rod 120 includes a thumb press 123 for a user to apply distally directed force to the thumb press 123 to expel fluid 140 from the syringe barrel 102.

Advantageously, in the embodiments shown, the elongate plunger rod 120 comprises four ribs 120a, 120b, 120c, and 120d, which divide the elongate plunger rod 120 in cross-section in quadrants. The plunger rod locking slots 126 are on each of the four ribs 120a, 120b, 120c and 120d, and this arrangement facilitates easier locking of the elongate plunger rod without having to rotate the elongate plunger rod 120 because the locking element can be engaged with the nearest of the four ribs 120a, 120b, 120c, and 120d.

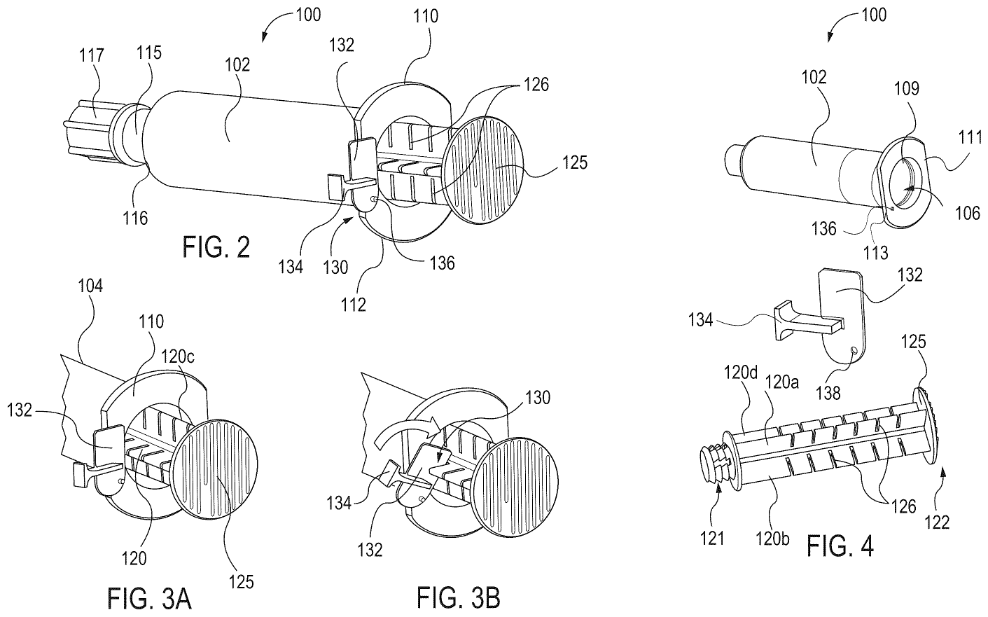

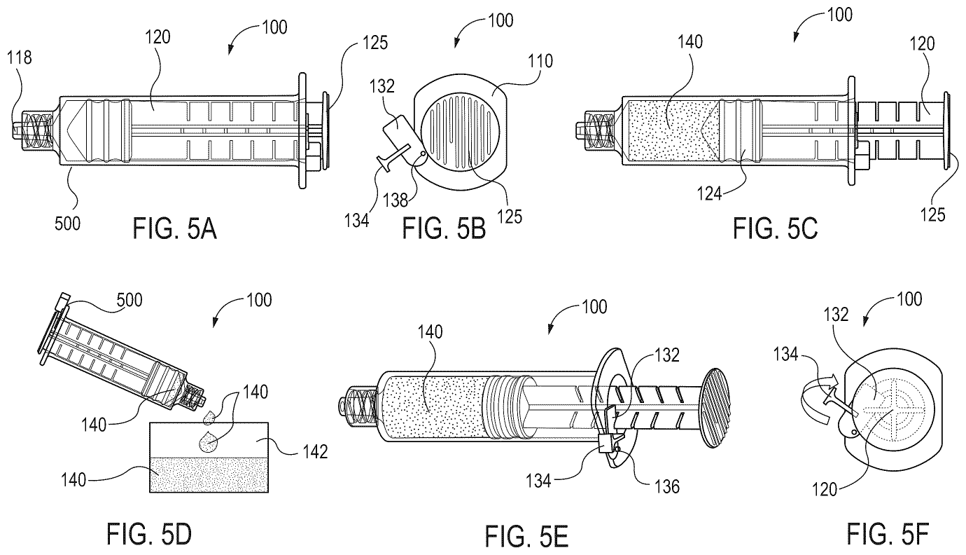

Referring to now to FIGS. 3A-F, FIG. 4, and FIGS. 5A-E, the locking element 130 comprises a pivotable locking tab 132 mounted to one of the pair of connecting flanges 111, 113 and configured to selectively engage each of the plurality of plunger rod locking slots 126 as the elongate plunger rod 120 is move in a distal direction to create a vacuum to aspirate fluid from a blood vessel and in a proximal direction to expel the fluid 140 from the syringe barrel 102.

The syringe assembly further comprises a pivot pin 136 extending from one of the pair of connecting flanges 111, 113 or from one of the pair of gripping flanges 110, 112, and the pivotable locking tab 132 further comprises an aperture 138 that engages the pivot pin 136 to secure the pivotable locking tab 132 to the open proximal end 109 of the syringe barrel 102. The aperture 138 is sized to friction fit or compression fit onto the pivot pin 136. FIG. 5D is a side elevation of the syringe assembly shown in FIG. 5C after the fluid 140 has been expelled from the syringe assembly 100 and into a container 142.

The pivotable locking tab 132 further comprising a finger support 134 extending from the pivotable locking tab 132, the finger support 134 configured to be engaged by a user of the syringe assembly to pivot the pivotable locking tab 132 between a locked position and an unlocked position as shown in FIGS. 5B-F. It will be appreciated that the syringe assembly shown is much easier to use by a practitioner. Locking an unlocking the elongate plunger rod into different plunger rod locking slots 126 along the length of the elongate plunger rod is accomplished by simply rotating the locking element by the user engaging the finger support 134 with a thumb or a finger to rotate the pivotable locking tab 132 about the pivot pin 136.

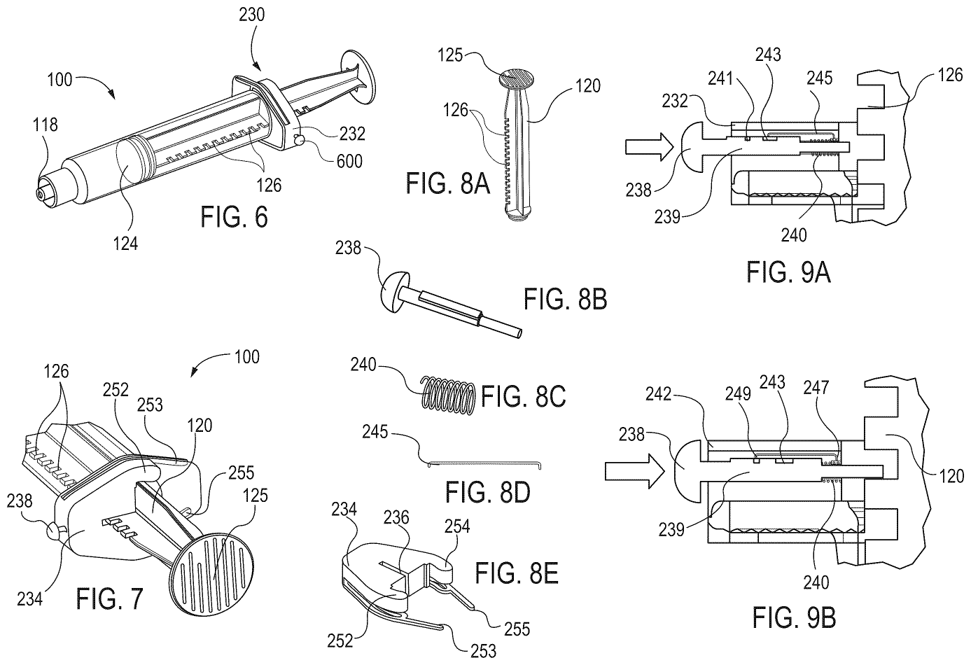

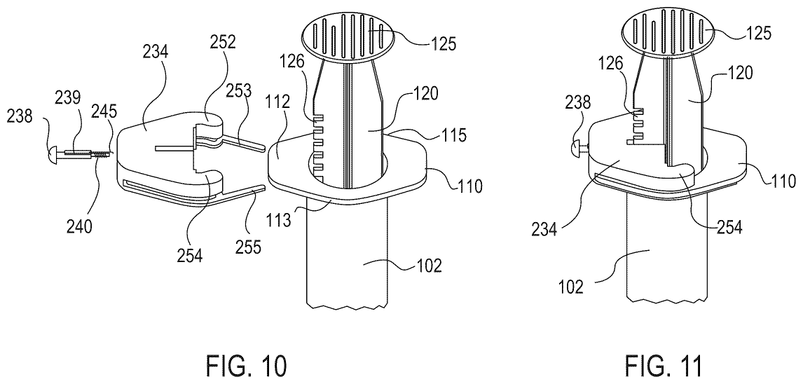

Referring now to FIGS. 6-11, a second embodiment of a syringe assembly 100 is shown. For ease of reference, the same reference numbers will be used in the different embodiments for the features of the syringe barrel 102, and the elongate plunger rod 120, and only the features of the locking element will utilize different reference numbers. Not all of the reference numbers are repeated for the syringe barrel 102 and the elongate plunger rod 120. As in the first embodiment, the syringe assembly 100 includes a syringe barrel 102 having a side wall 104 with an inside surface defining a chamber 106 configured to retain and expel a fluid, an open proximal end 109 having a pair of gripping flanges 110, 112 and pair of connecting flanges 111, 113 extending radially from the open proximal end 109 and a distal end 115 including a distal wall 116 having a passageway 118 therethrough in fluid communication with the chamber 106. The distal end 115 of the syringe barrel 102 includes suitable connector, which may be a Luer connector or other suitable connection configured to make a connection to a suitable devices such as tubing, a catheter or a system for removing fluid from a blood vessel.

As shown in FIGS. 6-11, the syringe assembly 100 further includes an elongate plunger rod 120 disposed within the syringe barrel 102, the elongate plunger rod 120 comprising a distal end 121 and a proximal end 122, the distal end 121 including a stopper 124, the elongate plunger rod including a plurality of plunger rod locking slots 126 located between the distal end 121 and the proximal end 122, and the elongate plunger rod 120 extendible to a plurality of different locked lengths using a locking element 130 mounted to the proximal end 109 of the syringe barrel 102 to engage the plurality of plunger rod locking slots 126 to permit varying amounts of fluid 140 to be aspirated into the syringe barrel 102 without rotating the elongate plunger rod 120. The proximal end 122 of the elongate plunger rod 120 includes a thumb press 123 for a user to apply distally directed force to the thumb press 123 to expel fluid 140 from the syringe barrel 102.

Referring now to FIGS. 6-11, the locking element 230 comprises a clip assembly 232 slidably mounted to the connecting flanges 111, 113, the clip assembly 232 comprising a main body 234 having a clip locking slot 236 that slidably engages the plurality of plunger rod locking slots 126 by a pushing force. The main body has a first extension arm 252 and first holding feature 253 attached to the first extension arm 252 and a second extension arm 254 spaced apart from the first extension arm 252 and a second holding feature 255 connected to the second extension arm 254. The extension arms 252, 254 and the holding features 253, 255 secure the clip assembly to the connecting flanges.

The clip assembly 232 further includes releasable locking pin 238 on an outer portion of the main body 234. The clip assembly 232 further includes a coil spring 240, and the releasable locking pin 238 is mounted within the coil spring 240 and slidably engaged within a receiving channel 242 in the main body 234. The releasable locking pin 238 has a releasable locking shaft 239 including a first detent 241 and a second detent 243 that cooperate with locator pin 245 that engages the releasable locking pin 238 at the first detent 241 and the second detent 243 in the receiving channel 242 respectively at an unlocked position and a locked position. The locator pin 245 has a first end having a finger 247 and a second end having a finger. The finger 249 at the send end of the locator pin 245 engages the first detent 241 and the second detent 243 of the releasable locking pin shaft 239 in respective unlocked and locked positions.

Application of a pushing force releases the finger 249 from first detent 241 and into the second detent place the releasable locking pin 238 in a locked position to engage a plunger rod locking slot as 127 shown in FIG. 9B. Application of a pushing force similarly releases the releasable locking pin 238, as the coil spring 240 biases the releasable locking pin from the elongate plunger rod locking slot 126 as shown in FIG. 9A.

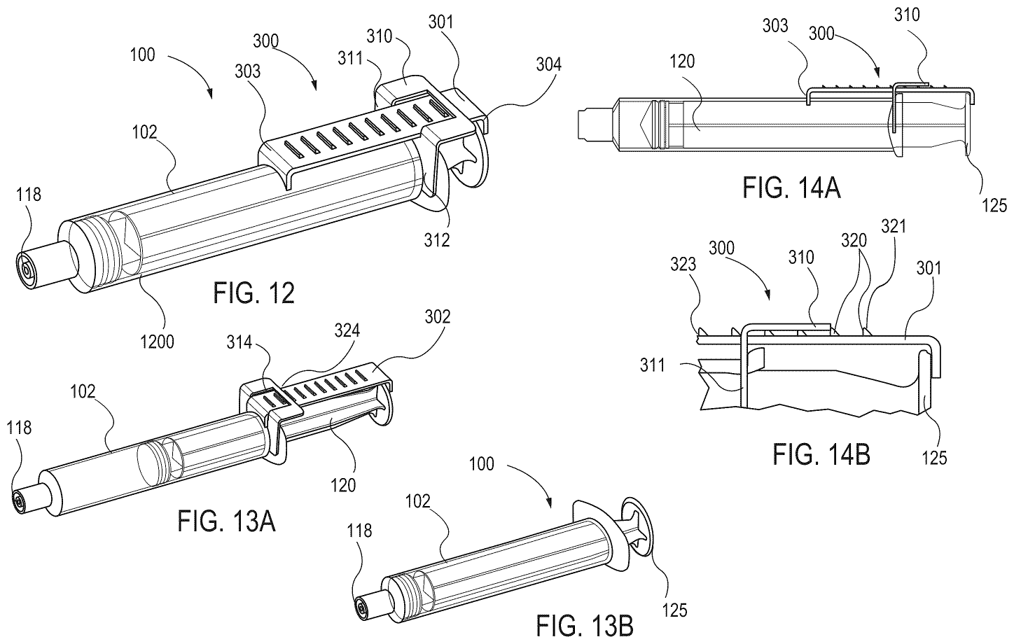

Referring now to FIGS. 12-14B, a third embodiment of a syringe assembly 100 is shown. For ease of reference, the same reference numbers will be used in the different embodiments for the features of the syringe barrel 102, and the elongate plunger rod 120 as used in the first and second embodiments of the syringe assembly 100, and only the features of the locking element will utilize different reference numbers. Not all of the reference numbers are repeated for the syringe barrel 102 and the elongate plunger rod 120. As in the first embodiment, the syringe assembly 100 includes a syringe barrel 102 having a side wall 104 with an inside surface defining a chamber 106 configured to retain and expel a fluid, an open proximal end 109 having a pair of gripping flanges 110, 112 and pair of connecting flanges 111, 113 extending radially from the open proximal end 109 and a distal end 115 including a distal wall 116 having a passageway 118 therethrough in fluid communication with the chamber 106. The distal end 115 of the syringe barrel includes a suitable connector, which may be a Luer connector or other suitable connection configured to make a connection to a suitable devices such as tubing, a catheter or a system for removing fluid from a blood vessel.

The syringe assembly 100 further includes an elongate plunger rod 120 disposed within the syringe barrel 102, the elongate plunger rod 120 comprising a distal end 121 and a proximal end 122, the distal end 121 including a stopper 124, the elongate plunger rod including a plurality of plunger rod locking slots 126 located between the distal end 121 and the proximal end 122, and the elongate plunger rod 120 extendible to a plurality of different locked lengths using a locking element 130 mounted to the proximal end 109 of the syringe barrel 102 to engage the plurality of plunger rod locking slots 126 to permit varying amounts of fluid 140 to be aspirated into the syringe barrel 102 without rotating the elongate plunger rod 120. The proximal end 122 of the elongate plunger rod 120 includes a thumb press 123 for a user to apply distally directed force to the thumb press 123 to expel fluid 140 from the syringe barrel 102

Referring now to FIGS. 12-14B, locking element 300 comprises an elongate bracket 302 including a distal end 303 and a proximal end 301 slidably mounted to the distal end of the syringe barrel 102. The distal end 303 of the elongate bracket 302 comprises a thumb press engagement tab 304 extending from the elongate bracket 302 and configured to engage the thumb press 125 and move with the thumb press 125 as the elongate plunger rod moves distally and proximally in the syringe barrel 102.

The locking element 300 further includes a mounting bracket 310 having a pair of syringe barrel engagement tabs 311, 312 that are spaced apart and configured that mount to the syringe barrel 102 adjacent to the pair of gripping flanges 112, 114 and an elongate bracket engagement slot 314 that permits the elongate bracket 302 to slidably move to a plurality of locked positions. The elongate bracket 310 has a plurality of spaced projections 320 configured to engage a stop element 324 on the mounting bracket 310. The stop element 324 is a crossbar that engages the spaced projections. Each of the spaced projections 320 comprising a ramped surface 321 and a stop surface 323. The stop surface 323 of each of the spaced projections 320 is perpendicular to the elongate bracket 302 to prevent distal motion of the elongate plunger rod 120 when in a locked position. The elongate bracket 310 is removable from the syringe barrel, as shown in FIG. 13B

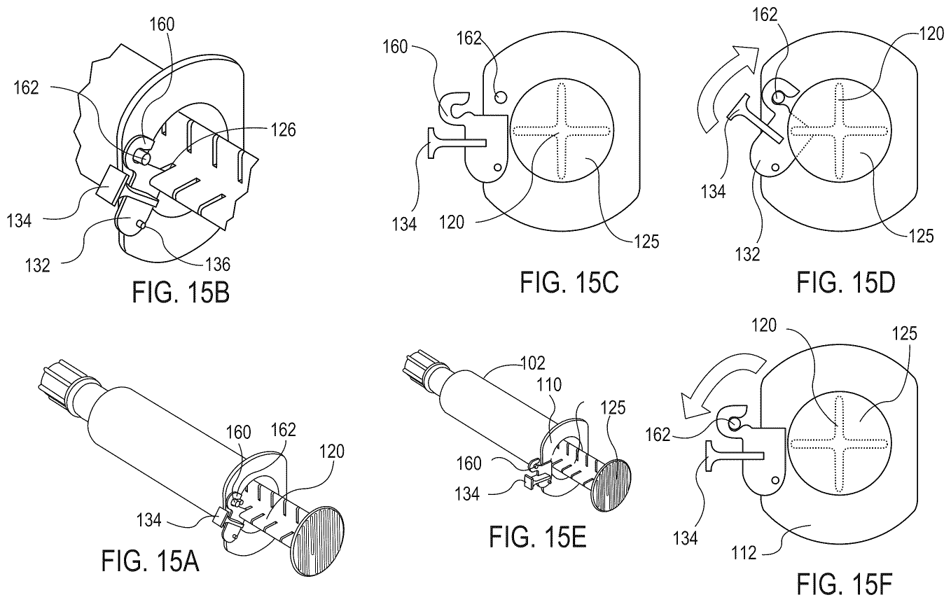

FIGS. 15A-B show a tamper-evident feature configured for use with the syringe assembly shown in FIG. 2. The tamper proof feature includes a hook 160 on the pivotable locking tab 132 that engages a breakable tamper pin 162 mounted to the proximal end of the syringe barrel. The breakable tamper pin 162 can be affixed to one of the pair of gripping flanges 110, 112, or to the one of the pair of connecting flanges 111, 113. In use, a syringe assembly 100 is provided to user as shown in FIG. 15D. If the breakable tamper pin 162 is removed from the proximal end of the syringe barrel 102 that the syringe assembly has been tampered with as shown in FIGS. 15 E and 15 F. The syringe assembly cannot be used because the breakable tamper pin 162 has been captured by the hook 160 and the breakable tamper pin 162. The hook 160 can no longer be locked to the tamper evident pin 162 as shown in FIG. 15F.

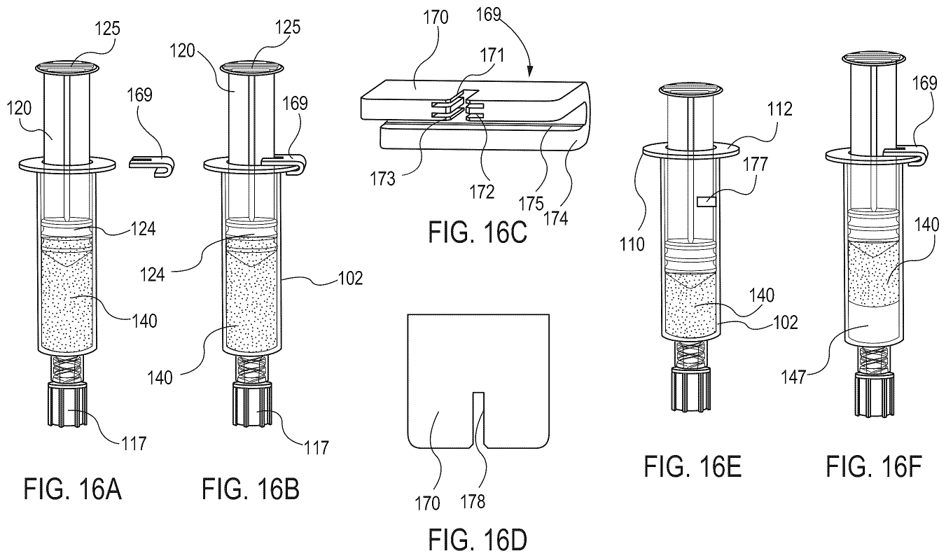

Referring now to FIGS. 16A-F, an embodiment of another type of tamper proof feature is shown. The tamper evident (or tamper proof) feature comprises a marking dye 177 placed on a clip, which in some embodiments, includes the clip locking slot 236 shown with respect to FIGS. 6-11. The marking dye 177 marks the elongate plunger rod 120 when the clip assembly is engaged with the elongate plunger rod to indicate the syringe assembly has been used. FIGS. 16A-F show a modification of a tamper clip 169, including a first leg 170 and a second leg 174 defining a recess 175 that allows the tamper clip 169 to be slidably mounted to one of the pair of gripping flanges 110, 112.

The first leg 170 comprises a first lip 171, a second lip 172 and a third lip 173 which define a clip locking slot 178, similar to the clip locking slot 236 shown with respect to FIGS. 6-11. The marking dye is applied to the second lip 172, which is disposed between the first lip 171 and the third lip 173. The tamper clip 169 is applied to the syringe barrel as shown in FIG. 16 B. FIG. 16E shows the elongate plunger rod 120 advanced in the syringe barrel 102 and leaving the marking dye 177 on the syringe barrel, indicating that the syringe has been used.

In the embodiment shown in FIGS. 16A-F, for demonstration of how the marking dye 177 marks movement of the elongate plunger rod 120 within the syringe barrel 102, the syringe is shown as having fluid 140 in the syringe barrel 102. After the tamper clip 169 has been removed, as shown in FIG. 16E, the marking dye 177 is left below the level of the pair of the gripping flanges 110, 112. When the plunger is pressed to expel the fluid 140 from the syringe barrel 102, the marking dye 177 provides evidence that the syringe has been tampered with. If the tamper clip 169 is reapplied after pulling the elongate plunger rod 120 back to match the marking dye 177, then the syringe will not be filled with the fluid 140, and there will be a free space 147, which provides an indication that some of the fluid 140 has been removed from the syringe barrel 102. According to one or more embodiments, the user would be aware from the tamper evident feature provided in FIGS. 16A-F if the syringe has been tampered with. This feature is useful for the vacuum lock syringes when used for extraction of material from a patient as further described herein. In addition, the vacuum lock syringes described herein can also be used in a pharmacy where the fluid 140 in the syringe barrel 102 is a drug product. The tamper evident feature described with respect to FIGS. 16A-F can be used as an indicator to restrict inadvertent plunger rod movement during handling.

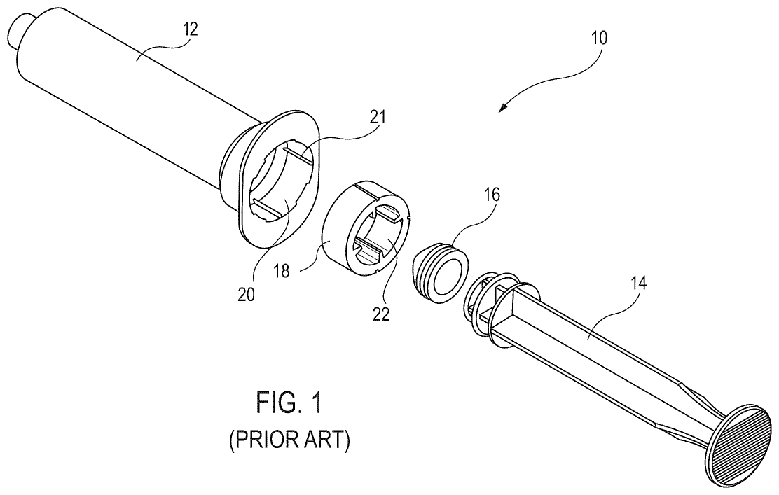

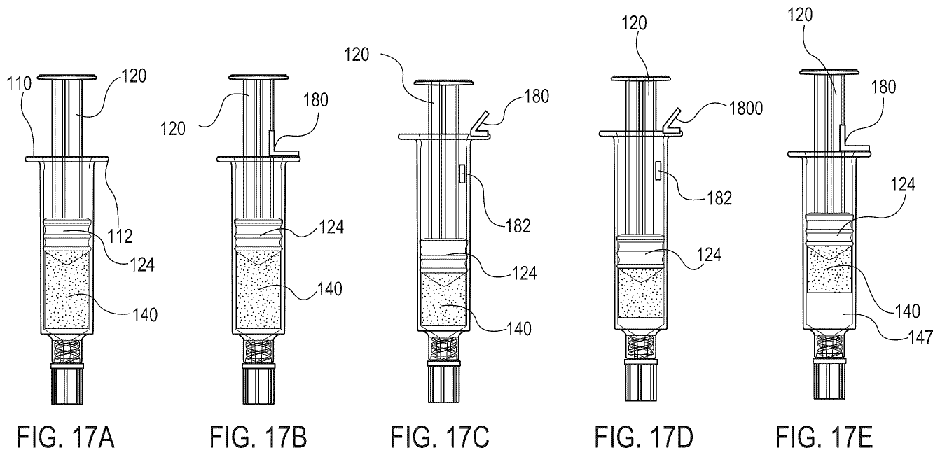

Referring now to FIGS. 17A-E a side elevation view of a tamper evident label for a syringe assembly. The syringe assemblies described herein can include a tamper evident feature comprising an adhesive label 180 attached to one of the pair of the gripping flanges 110, 112 and the elongate plunger rod 120, which breaks when plunger rod 120 has been moved, as shown in FIGS. 17C-D. A shown in FIGS. 17C-D, a label impression 182 is left on the elongate plunger rod 120. As shown in FIG. 17D, if the label 180, which has been broken, is reapplied without pulling the elongate plunger rod 120 back in a proximal direction, the label impression is visible below the level of the pair of gripping flanges 110, 112, providing evidence that the syringe has been tampered with. As shown in FIG. 1E, if the label 180 is reapplied after pulling the elongate plunger rod 120 back to match the impression, then the syringe will not be filled with fluid and there will be a free space 147, indicating that the syringe has been tampered with.

According to one or more embodiments, the user would be aware from the tamper evident feature provided in FIGS. 17A-E if the syringe has been tampered with. This feature is useful for the vacuum lock syringes when used for extraction of material from a patient as further described herein. In addition, the vacuum lock syringes described herein can also be used in a pharmacy where the fluid 140 in the syringe barrel 102 is a drug product. The tamper evident feature described with respect to FIGS. 17A-E can be used as an indicator to restrict inadvertent plunger rod movement during handling.

According to one or more embodiments, described herein, syringe assemblies are provided that are configured for use as vacuum pressure syringe that can be utilized in a variety of clinical procedures where a vacuum pressure syringe is required to aspirate material such as fluid, plaque, clots, etc. from blood vessels of a patient. For example, the syringe assemblies can be to aspirate coronary plaque during an atherectomy, removal of an abscess, thrombus and coronary plaque drainage.

In each of the embodiments, described herein, the elongate plunger rod comprises slots and a locking feature that allows a user to retract the elongate plunger rod from the syringe barrel to create a sustained vacuum and to lock the elongate plunger rod in place a one of various desired locations along the length of the elongate plunger rod. The syringe assemblies described according to one or more embodiments can be attached to a stopcock or a tubing set/catheter to perform the desired medical procedure.

Vacuum is created by withdrawing the elongate plunger rod from the syringe barrel. Then, without having the rotate the elongate plunger rod, a user of the device can lock the device in place. In one embodiment, the user moves or swings a locking tab about a pivot to interlock the pivotable locking tab with a slot along the length of the elongate plunger rod. In another embodiment, the locking element to lock the elongate plunger rod in place comprises a slidably mounted clip assembly that the user engages and disengages by pressing a pin on the clip assembly. In another embodiment, the elongate plunger rod is locked in place by a slidable bracket that is removable from the syringe.

After the elongate plunger rod has been locked in place, a sustained vacuum is obtained by the syringe assembly to assist in desired material or fluid removal. In some embodiments, the elongate plunger rod comprises ribs, and there are slots in all of the ribs, Hence, the locking procedure does not depend on the orientation of rib with respect to the locking element. In one or more embodiments, the locking element is configured to lock with any of the ribs in the elongate plunger rod, and rotation is not required to lock the elongate plunger rod in place.

For example, the pivotable locking tab can be rotated about the pivot to lock with a slot in the nearest rib of the elongate plunger rod. Inn some embodiments, the locking element is a separate attachment which can be attached to a typical syringe and a specially manufactured syringe is not required. The detachable locking element can be fitted to existing syringes.

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in various embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein provided a description with reference to particular embodiments, it is to be understood that these embodiments are merely illustrative of the principles and applications of the disclosure. It will be apparent to those skilled in the art that various modifications and variations can be made to the present disclosure without departing from the spirit and scope thereof. Thus, it is intended that the present disclosure include modifications and variations that are within the scope of the appended claims and their equivalents.

What is claimed is:

1. A syringe assembly comprising:
a syringe barrel having a side wall with an inside surface defining a chamber configured to retain and expel a fluid, an open proximal end having a pair of gripping flanges and pair of connecting flanges extending radially from the open proximal end and a distal end including a distal wall having a passageway therethrough in fluid communication with the chamber; and
an elongate plunger rod disposed within the syringe barrel, the elongate plunger rod comprising a distal end and a proximal end, the distal end including a stopper, the elongate plunger rod including a plurality of plunger rod locking slots located between the distal end and the proximal end, and the elongate plunger rod extendible to a plurality of different locked lengths using a locking element mounted to the proximal end of the syringe barrel to engage the plurality of plunger rod locking slots to permit varying amounts of fluid to be aspirated into the syringe barrel without rotating the elongate plunger rod.

2. The syringe assembly of claim 1, the locking element further comprising a pivotable locking tab mounted to one of the pair of connecting flanges and configured to selectively engage each of the plurality of plunger rod locking slots.

3. The syringe assembly of claim 2, further comprising a pivot pin extending from one of the pair of connecting flanges, and the pivotable locking tab further comprising an aperture that engages the pivot pin to secure the pivotable locking tab to the distal end of the syringe barrel.

4. The syringe assembly of claim 3, the pivotable locking tab further comprising a finger support extending from the pivotable locking tab, the finger support configured to be engaged by a user of the syringe assembly to pivot the pivotable locking tab between a locked position and an unlocked position.

5. The syringe assembly of claim 1, the locking element comprising a clip assembly slidably mounted to the pair of connecting flanges, the clip assembly comprising a main body having locking slot that slidably engages the plurality of plunger rod locking slots by a pushing force.

6. The syringe assembly of claim 5, the clip assembly further comprising releasable locking pin on an outer portion of the main body.

7. The syringe assembly of claim 6, the clip assembly further comprising a coil spring, and the releasable locking pin mounted within the coil spring and slidably engaged within a receiving channel in the main body.

8. The syringe assembly of claim 7, the clip assembly further comprising a locator pin that engages the releasable locking pin in the receiving channel at a locked position and an unlocked position.

9. The syringe assembly of claim 8, the locator pin having first finger at a first end and a second finger at a second end that engages first detent and a second detent on the releasable locking pin.

10. The syringe assembly of claim 9, wherein application of a pushing force releases the first finger from the first detent to unlock the releasable locking pin from the locked position.

11. The syringe assembly of claim 1, the locking element comprising an elongate bracket including a distal end and a proximal end slidably mounted to the distal end of the syringe barrel.

12. The syringe assembly of claim 11, the distal end of the elongate bracket comprising a thumb press engagement tab extending from the elongate bracket.

13. The syringe assembly of claim 12, further comprising a mounting bracket having a pair of syringe barrel engagement tabs that mount to the syringe barrel adjacent to the pair of gripping flanges and an elongate bracket engagement slot that permits the elongate bracket to slidably move to a plurality of locked positions.

14. The syringe assembly of claim 13, the elongate bracket having a plurality of spaced projections configured to engage a stop element on the mounting bracket.

15. The syringe assembly of claim 14, each of the spaced projections comprising a ramped surface and a stop surface.

16. The syringe assembly of claim 15, wherein the stop surface of each of the spaced projections prevents distal motion of the elongate plunger rod.

17. The syringe assembly of claim 16, wherein the elongate bracket is removable from the syringe barrel.

18. The syringe assembly of claim 2, further comprising a tamper evident feature including a hook that engages a breakable tamper pin mounted to the proximal end of the syringe barrel.

19. The syringe assembly of claim 5, further comprising a tamper evident feature including a marking dye on the locking slot that marks the elongate plunger rod when the clip assembly is engaged with the elongate plunger rod to indicate the syringe assembly has been used.

20. The syringe assembly of claim 1, further comprising a tamper evident feature comprising an adhesive label attached to one of the pair of the gripping flanges and the elongate plunger rod, which breaks when plunger rod has been moved.
